(19) United States
(12) Patent Application Publication
(10) Pub. No.: US 2022/0343882 A1
(43) Pub. Date: Oct. 27, 2022

(54) METHOD AND SYSTEM FOR TRANSLATION OF BRAIN SIGNALS INTO ORDERED MUSIC

(71) Applicants: Brian Forbes, Brooklyn, NY (US); Maria Genesis-Brodskaya, Brooklyn, NY (US)

(72) Inventors: Brian Forbes, Brooklyn, NY (US); Maria Genesis-Brodskaya, Brooklyn, NY (US)

(21) Appl. No.: 17/727,733

(22) Filed: Apr. 23, 2022

Related U.S. Application Data
(60) Provisional application No. 63/178,937, filed on Apr. 23, 2021.

(51) Int. Cl. G10H 1/00; A61B 5/369

(52) U.S. Cl. CPC G10H 1/0025; A61B 5/369; G10H 2210/101

(57) ABSTRACT

The present invention is a computer-implemented method comprising: receiving, by one or more processors, data from an electroencephalogram device worn by a user, wherein data is collected related to at least one brainwave; separating the collected data into individual data streams related to the one or more brainwaves; performing at least one manipulation to each of the individual data streams, wherein each of the data streams are manipulated to produce a sound applying at least one filter to each of the sounds; and generating each of the sounds, wherein a musical composition is formed.

### CROSS-REFERENCE TO RELATED APPLICATIONS

This application is a continuation-in-part (and claims the benefit of priority under 35 USC 120) of U.S. application No. 63/178.937 filed Apr. 23, 2021. The disclosure of the prior applications is considered part of (and is incorporated by reference in) the disclosure of this application.

### BACKGROUND

This disclosure relates generally to the creation of music or musical sounds, and more specifically to a method, computer program and computer system that is designed for the creation of the music from data collected from a person's brainwaves.

Electroencephalogram (electroencephalogram, EEG) is a kind of technology for monitoring electrical brain activity. It is widely used in clinical and scientific research. A human brain generates bio-signals such as electrical patterns, which may be measured or monitored using an electroencephalogram (EEG). Typically, an EEG will measure brainwaves in an analog form. Then, these brainwaves may be analyzed either in their original analog form or in a digital form after an analog to digital conversion is performed. This converted form can be used to create both visual and audible creations.

Brainwave music, which is simply music derived from brainwaves, contains physiological information of the brain. In recent years, related researchers at home and abroad propose various methods based on different theories to convert a single-lead or multi-lead electroencephalogram (EEG) signal and a functional magnetic resonance signal (FMRI) synchronously acquired with the EEG into music, analyze and apply the obtained music, and show a new way to explore the relation between the brain and the music.

Music is originally a distinct field from information technology and biomedical engineering, while brainwave music combines music with biomedical engineering. At present, the research on brainwave music is that the characteristics of the frequency, amplitude, and the like, of original brainwaves are directly mapped to the duration and pitch of music, and the generated music depends on the biological signals of a human body and can reflect the current state of a user. Since brainwave music is always in lack of an authoritative mode in the mapping relation between biological signals and music, and the fact that most researchers are not skilled at generating music, brainwave music obtained by the prior art is relatively stiff and lacks artistry.

AI (Artificial Intelligence) music is music generated by an algorithm that relates music to information technology. Along with the development of artificial intelligence, the research of AI music is receiving more and more attention, but basically, the music composition is carried out by a computer in a mode of building a model or training a neural network by a large number of music pieces. Although AI music can obtain artistic music under the development background of machine learning, the current research is basically on computer music composition, and the AI music generated by the prior art largely depends on model architecture and training data and lacks real-time human-computer interactivity.

Current audio-based EEG products only use the neurofeedback to effect certain parameters of pre-recorded material such as volume, panning or LPF/HPF-type filtering EQing. Or to trigger certain sounds when the info stream passed a certain threshold.

However, the present music generation tools and methods fail to provide music using all aspects of the brainwaves which are collected and also fail to provide music that has artistic value, pleasing sounds, or efficient use of the data collected. Therefore, brainwave music and AI music generated based on the prior art have disadvantages, and an AI music generation technology which can realize real-time brainwave control and is artistic is lacked at present.

### SUMMARY

In a first embodiment, the present invention is a computer-implemented method comprising: receiving, by one or more processors, data from an electroencephalogram device worn by a user, wherein data is collected related to at least one brainwave; separating the collected data into individual data streams related to the one or more brainwaves; performing at least one manipulation to each of the individual data streams, wherein each of the data streams are manipulated to produce a sound; applying at least one filter to each of the sounds; and generating each of the sounds, wherein a musical composition is formed.

In a second embodiment, the present invention is a computer program product comprising: a computer non-transitory readable storage medium having program instructions embodied therewith, the program instructions executable by a computing device to cause the computing device to: receiving data from an electroencephalogram device worn by a user; decoding the data received by the electroencephalogram device into individual brainwaves; associating each of the individual brainwaves to a sound or to an effect of a sound; performing a translation to each of the individual brainwaves collected data; applying a plurality of manipulations to the translated data for each of the individual brainwaves; generating a series of sounds from the manipulated translated data from the individual brainwaves associated with a sound, and wherein the individual brainwaves which are associated with an effect of a sound are applied to the corresponding manipulated translated data from the individual brainwaves associated with a sound; and outputting, a musical composition from the series of sounds.

In a third embodiment, the present invention is a system comprising: a CPU, a computer readable memory and a computer non-transitory readable storage medium associated with a computing device; receiving data from an electroencephalogram device worn by a user; decoding the data received by the electroencephalogram device into individual brainwaves; associating a first set of brainwaves to a sound and a second set of brainwaves to an effect; performing a translation of each of the brainwaves into data; associating the second set of brainwaves associated with an effect to the first set of brainwaves associated with a sound; applying the effects to the associated brainwave, wherein a modified sound is created; and generating a musical composition from the manipulated sounds.

### BRIEF DESCRIPTION OF THE DRAWINGS

Referring now to the drawings in which like reference numbers represent corresponding parts throughout:

FIG. 1 depicts a cloud computing node according to an embodiment of the present invention.

FIG. 2 depicts a cloud computing environment according to an embodiment of the present invention.

FIG. 3 depicts a block diagram depicting a computing environment according to an embodiment of the present invention.

FIG. 4 depicts a flowchart of the operational steps taken by a program to convert the brainwaves into ordered music using a computing device within the computing environment of FIG. 1, according to an embodiment of the present invention.

FIGS. 5A and 5B depicts another flowchart of the operational steps taken by the program to convert the brainwaves into ordered music using a computing device within the computing environment of FIG. 1, according to an embodiment of the present invention.

FIG. 6 depicts a user interface showing the ordered music, according to an embodiment of the present invention.

FIG. 7 depicts a user interface showing the received brainwaves, according to an embodiment of the present invention.

FIG. 8 depicts a user interface showing a visual product created by the program based on the received brainwaves, according to an embodiment of the present invention.

### DETAILED DESCRIPTION

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects may generally be referred to herein as a “circuit,” “module”, or “system.” Furthermore, aspects of the present invention may take the form of a computer program product embodied in one or more computer readable medium(s) having computer readable program code/instructions embodied thereon.

The present invention is an AI program that translates EEG signals from the brain in real-time into ordered musical information and performs this music on various digital instruments as to simulate a unique human music composition and performance for listening, meditation or neurofeedback exercise. A problem was identified when first approaching the question of turning brainwaves into music. There were no examples of anyone making anything truly listenable or “musical” from EEG info. We were also frustrated with the limitations of interactive neurofeedback within meditation apps designed for EEG headbands.

The present invention uses a developed artificial intelligence, or machine learning system that takes the data output of an EEG headset or headband and translates it in real-time into melodic and harmonic counterpoint that simulates a unique human musical composition. Each brainwave (alpha, beta, delta, gamma, theta) can have a musical representation as either a single or a multi-voice musical instrument created by an onboard synthesizer or sampler. the user can choose customized sound sets for both meditation and listening/aesthetic pleasure, or the user can choose sound sets that are designed for use in neurofeedback exercises that can help with attention deficit, anxiety, self-love, self-improvement (e.g., quit smoking, alcohol use or drug use, etc.), depression or other issues. In some of these settings, the invention may take a self-hypnosis design or functionality. In some embodiments, the invention may be beneficial in that listening to music created by one's own brain is used as therapy or partial therapy for mitigating or preventing Alzheimer' s or/and other types of dementia, improving brain-activity, enhancing cognitive functionality and brain cell connections, increasing the resiliency of the brain, reducing agitation and improving behavioral issues. In another embodiment, the music created can be used for studying and work, in schools and in corporate environment, since the music generated is never repeating and endless (as long as one is wearing a device and using the technology) music track helping to concentrate, focus, which is improving the productivity of work and study.

In some embodiments, the invention may take on an ability to allow the user to relive moments, as the music that is created is in the form of a moment in time or moments in their life that are now manifested into a recording, allowing the user to relive these moments through the music. Using the present technology and creating customized sound sets, the customers' memories and emotions that are represented by their brain-activity are translated into music and recorded, forming literal sonification of presented brain-activity as the Open Sound Control (OSC) stream. It can also be applied in settings to capture and record the brain activity of terminally sick or older family members, in order to sonificate their personality, their mind and soul via recoded brain-activity by the OSC stream, using the present technology, and then translating it into a musical piece.

The music created by the present invention is able to be transcribed or written to allow a person to play (or perform) the generated music. This can be done by an ensemble, orchestra, choir, or the like.

The present invention creates the audio material-musical composition in its entirety from the information stream provided by the EEG. There are at least 5 unique sets of melodic and/or harmonic information, one for each type of brainwave (alpha, beta, delta, gamma, theta) and assign each channel an instrument or voice. The user can then mix their own experience choosing their volume level of all combinations of the 5 music streams. This audio material is unique every time as they are purely based on the input of the EEG brainwaves. This piece of music can be used for entertainment, or for contemplative meditation of/for direct neurofeedback training.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods, and computer program products according to various embodiments of the present invention. In this regard, each block in the flowcharts may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the flowchart illustrations, and combinations of blocks in the flowchart illustrations, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

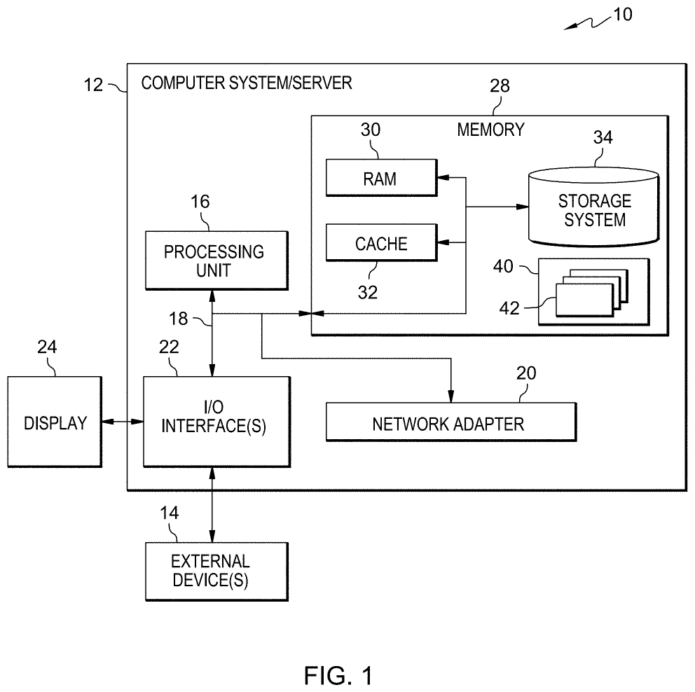

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random-access memory (RAM) 30 and/or cache memory 32. Computer system/server 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to microcode, device drivers, redundant processing units, external disk drive arrays, RAID systems, tape drives, and data archival storage systems, etc.

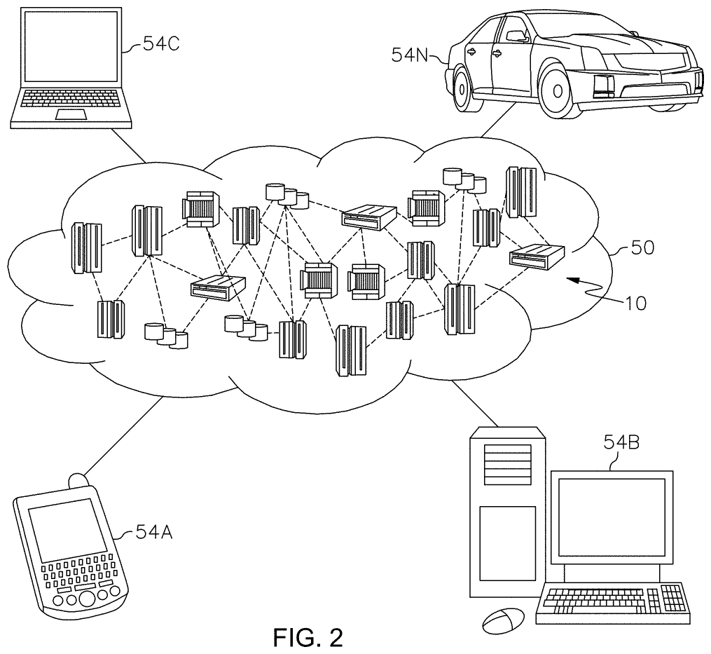

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, and laptop computer 54C may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-C shown in FIG. 2 are intended to be illustrative only and that computing nodes 10 and cloud computing environment 50 can communicate with any type of computerized device over any type of network and/or network addressable connection (e.g., using a web browser).

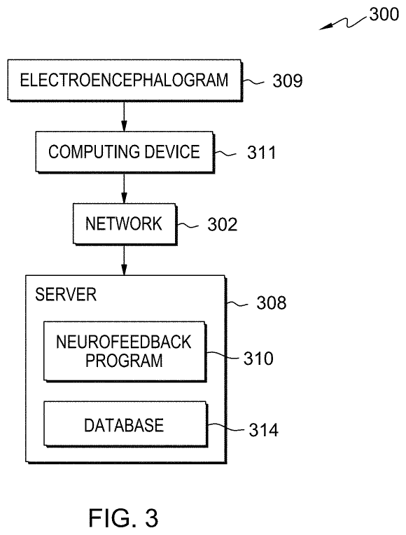

Referring back to FIG. 1, the Program/utility 40 may include one or more program modules 42 that generally carry out the functions and/or methodologies of embodiments of the invention as described herein. Specifically, the program module 42 is able to collect data from an electroencephalogram machine (EEG) or the like which is able to collect data related to a wearers brain activity and convert this data into ordered music or sounds. The collected brain activity data is processed to associate each brainwave (alpha, beta, delta, gramma, theta, etc.) to a specific instrument, sound, or the like which in combination with the other brainwaves creates ordered music. This can be used in both a therapeutic setting or to accompany performances. For example, in a therapeutic season, the music, which is created by the wearer, can be used to relax them, and in a performance situation, the instruments or sounds generated by the performer can be used to accompany a performance (e.g., musical, vocal, etc.) to enhance the performance. Other functionalities of the program modules 42 are described further herein such that the program modules 42 are not limited to the functions described above. Moreover, it is noted that some of the modules 42 can be implemented within the infrastructure shown in FIGS. 1-3. For example, the modules 42 may be representative of a parking selection server as shown in FIG. 3.

FIG. 3 depicts a block diagram of a computing environment 100 in accordance with one embodiment of the present invention. FIG. 3 provides an illustration of one embodiment and does not imply any limitations regarding the environment in which different embodiments maybe implemented.

Network 102 may be a local area network (LAN), a wide area network (WAN) such as the Internet, any combination thereof, or any combination of connections and protocols that can support communications between the components of the environment.

Electroencephalogram machine (EEG) 109 is a device that records the electrical activity of the brain. It contains electrodes that can detect brain activity when placed on a subject's scalp. The electrodes record the brainwave patterns, and the EEG 109 sends the data to a computer or cloud server.

Computing device 111 may be a smart phone, mobile phone, laptop, or any other electronic device or computing system capable of processing program instructions and receiving and sending data via network 102.

Server 108 may be a management server, a web server, or any other electronic device or computing system capable of processing program instructions and receiving and sending data. In other embodiments server 108 may be a laptop computer, tablet computer, netbook computer, personal computer (PC), a desktop computer, or any programmable electronic device capable of communicating via network 102. In one embodiment, server 108 may be a server computing system utilizing multiple computers as a server system, such as in a cloud computing environment. In one embodiment, server 108 represents a computing system utilizing clustered computers and components to act as a single pool of seamless resources. In the depicted embodiment database 114 and neurofeedback program 110 are located on server 108. Server 108 may include components, as depicted and described in further detail with respect to FIG. 1.

Neurofeedback program 110 processes the data collected from the EGG 109 and converts it to music through a series of translations, manipulations, and filtering of the received data.

Database 114 may be a repository that may be written to and/or read by. Information gathered may be stored to database 114. Such information may include previous scores, audio files, textual breakdowns, facts, events, and contact information. In one embodiment, database 114 is a database management system (DBMS) used to allow the definition, creation, querying, update, and administration of a database(s).

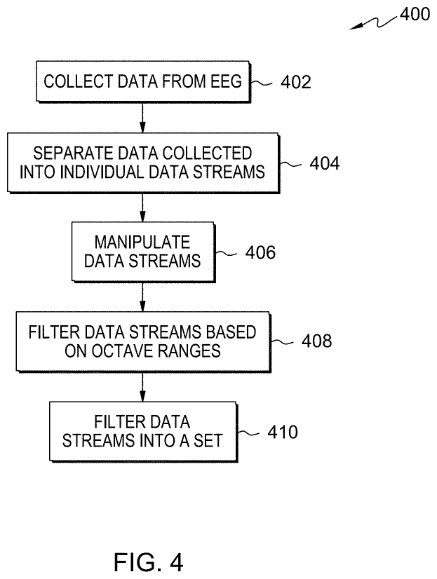

FIG. 4 shows flowchart 400 depicting a method according to the present invention. The method(s) and associated process(es) are now discussed, over the course of the following paragraphs, with extensive reference to FIG. 2, in accordance with one embodiment of the present invention.

The program(s) described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The program provides for the generation of music or sounds from a person's brainwaves. The music or sound is based on the number of brainwaves which are collected by the EEG machine, and the program's processing of each individual brainwave and what sound, instrument, or music is associated with that brainwave. For example, each brainwave may be associated with a different instrument (e.g., guitar, violin, harp, flute, drum, etc.), voice, sound, or noise, or may be associated with a group of instruments, sounds, or voices (hereinafter referred to as sounds). In other embodiments, the program may have predetermined brainwaves associated with sounds and other brainwaves associated with adding effects to these sounds. For example, if the alpha brainwave is associated with a guitar, the beta brainwave can be associated with an effect on that guitar which modifies the frequency range, modulation effects, and the like.

This provides a novel way to both generate music or sounds to accompany music or performances. Given the multiple brainwaves which can be analyzed using an EEG machine (or similar device), a multitude of sounds can be generated with nothing more than a user's brain. The applications of the present invention can be used in both a therapeutic setting as well as a performance setting. The music which is created by the user can be used in combination with the user playing another instrument or performing alongside the generated music or sounds. The sounds generated by the program 110 can act as a solo performer or may be used to accompany a person performing. While one example of this would be a violinist playing with the music created by the program acting as additional sounds to the piece. This can also be used say for an artist to create music or sounds while they paint or create their art, or a dancer performing with the program creating the music which the dancer is performing too. In a therapeutic setting, the program can create music or sounds which can assist the person to relax or to enhance the therapeutic process by providing a subconscious cue or indicator to the session. In an additional setting, the program can take the collected brainwave activity and instead of converting the data to sounds, can convert the data to visuals (either still or moving).

The present invention also provides an advantage of how performances, therapy, and a variety of other activities can be performed with the ability to take the user's brainwaves and convert them to a visible or audible medium. Providing insight into the person's subconscious.

In some embodiments, the program 110 receives a set of criteria that is used to determine a purpose for the process. This criteria is related to the purpose of the experience for the user. This may be included in a form or information provided by the user which the program 110 processes to adjust the overall settings of the process. In other embodiments, this information may be selected by the program 110 based on a desired end result. For example, the information provided by the user may be, but not limited to, information about the user's dreams, likes, dislikes, recent experiences, music preferences, the therapeutic objective of the session, enhancement of activities, and the like. The program 110 uses this information to determine the number of instruments, sounds, or voices (hereinafter referred to as sounds) to determine if the created music is solo, duo, trio, or another grouping of sounds, the pairing of sounds which can be selected, major or minor keys, electronic or acoustic, sound sets based on psychological goals, and the like. In some embodiments, sound sets are predetermined and include set sounds This step determines the overall trajectory of the session.

This selection based on the criteria also determines the number of different sounds, the brainwave the sound is associated with, and also at least one brainwave is associated with an effect, and which sound the effect is applied to. For example, alpha brainwave may be assigned a violin and beta brainwave may be associated with a filter that is applied to the violin.

In step 402, the program 110 collects data from the EEG machine 109. The OSC stream output from an EEG machine 109 is collected and provided to the computing device 111. In some embodiments, additional devices are used to collect jaw movement, eye movement, and other body movements and other recordable data which the user's body. In these embodiments additional devices may be required to collect these additional data sets, such as an accelerometer or the like. In one embodiment, the collection of the data is performed in real time.

In some embodiments, music or sound is played for the user to encourage certain brain responses. For example, binaural beats are played to the user while wearing the EEG. These encourage certain brainwaves in the user. By generating a tone in the left ear that differs from the right by the wavelength of a theta wave is used to induce more theta wave activity in the user. This can be done to induce more of one or more brainwave and may be integrated into the process at predetermined times or intervals.

In step 404, the program 110 separates the collected data into distinct components. The program 110 is able to take sort the brain activity collected by the EEG machine 109 and convert it into individual brainwaves (e.g., alpha, beta, delta, gamma, theta, etc.). In some embodiments, the EEG machine 109 is able to collect and convert the brain activity into individual brainwaves. The number of brainwaves is dependent upon the EEG machines 109 ability, the programs 110 ability, or the requested brainwaves by the program 110. This is typically the five different brainwaves (alpha, beta, delta, gamma, theta).

In step 406, the program 110 takes the data points associated with the individual brainwaves and converts/manipulates them to a digital formation that is then used to create a note. The note is based on the sound selection, so for example the notes would be made to sound like a violin if the brainwave is assigned a violin. Given that the data collected by the EGG machine 109 is a plurality of data points, the program 110 is constantly receiving data related to each brainwave and the shift in the frequency of the brainwave. The changes in the value of each brainwave affect the note which is created by the program 110. In some embodiments, the program 110 only receives data when it is above a predetermined value. In some embodiments, the program 110 uses one brainwave for the sound and another brainwave as an effect on the sound, the OSC stream of the brainwave associated with the effect changes the intensity or degree the effect plays on the note. For example, alpha may be related to a guitar and beta may be associated with the pitch of the guitar, so the data collected related to alpha may affect the note the guitar makes, and beta affects the pitch of the note. In some embodiments, the program 110 uses the OSC stream to determine the velocity of the note.

In step 408, the program 110 filters the collected data and manipulates the collected data to create the effects and filters which are associated with the notes. The collected data is filtered to remove values above and below predetermined values so that a range of frequencies of the brainwaves is collected. The program 110 can also manipulate the collected data so that notes are brought within an audible range or a more ideal range. For example, where notes are below C2, these notes are raised up by two or three octaves, and notes above C7 are lowered by two or more octaves. The parameters for what the high and low ranges of the notes, and also how far the notes are adjusted ca be set by the program 110 or manually.

In some embodiments, the program 110 adjusts the notes so that they are set in one key. The key is the group of pitches, or scales, which form the basis of the musical composition. Given the different brainwaves and frequencies, the values collected may result in the notes spanning many keys. However, in typical music compositions this is not ideal and thus the collected data needs to be adjusted to fit within one key. The key may be predetermined or selected using artificial intelligence or machine learning technology based on input data and previously collected data. In some embodiments the key is seven (7) or five (5) note scales (major, minor, pentatonic etc.). In some embodiments, the program 110 creates the note envelope or the attack, decay, sustain, and release (ADSR) of the notes or sound sets. Based on the note envelope, the program 110 is able to determine when the note or sound set is turned on or brought into the final musical composition.

In step 410, the program 110 filters the data streams into a set and produces the musical composition. The collected and processed data that was manipulated through the preceding steps is fed through a synthesizer or a sampler for playback. This process can be recorded and played back or can be live. In some embodiments, the notes produced by the collected data is paired with another OSC stream that will create polyphonic chords. In some embodiments, there is a mixer element which controls the level of each of the OSC streams.

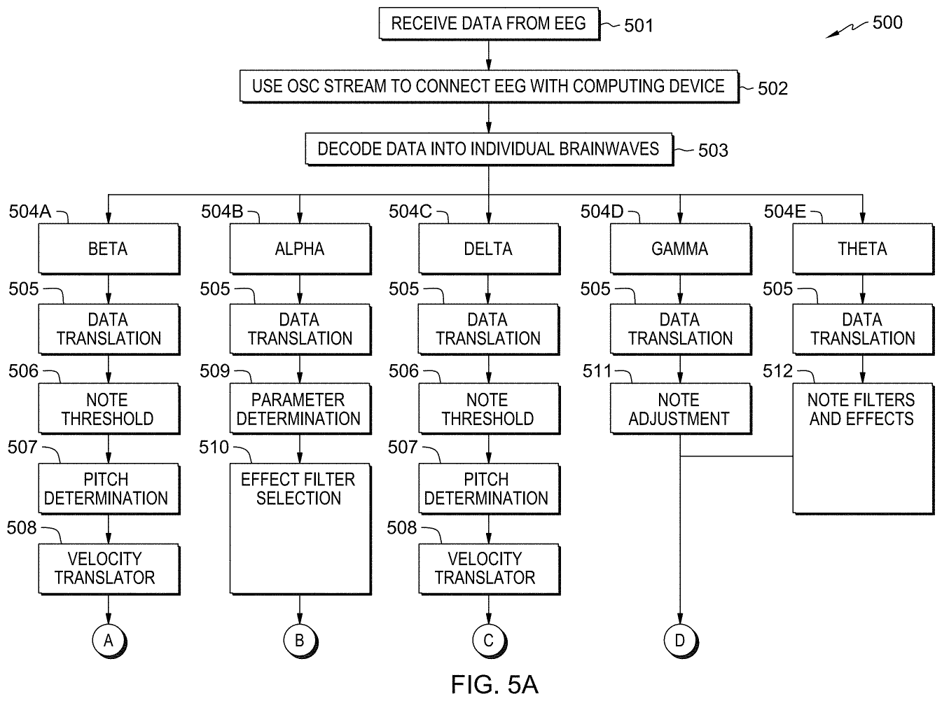

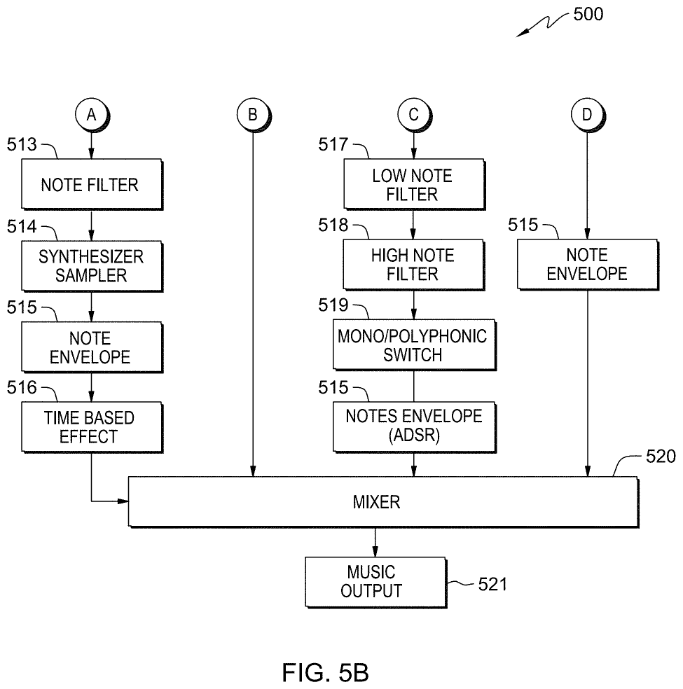

FIG. 5 depicts flowchart 500 of the operational steps taken by a program to convert the brainwaves into ordered music using a computing device within the computing environment of FIG. 1, according to an embodiment of the present invention.

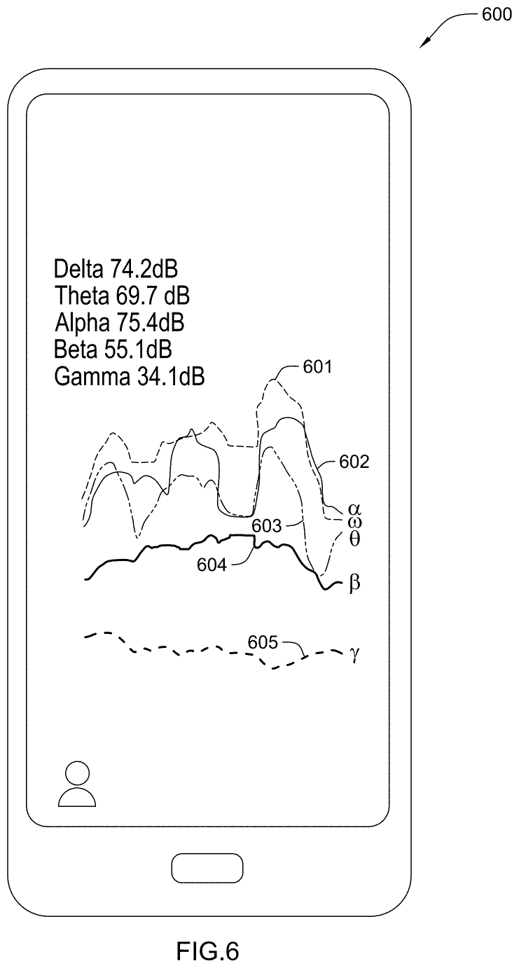

In the depicted embodiment, the EEG machine 109 is worn by the person, and data is continuously collected by the EEG machine 109 (step 501) which forms the OSC stream (step 502) which is transmitted to a computing device. The OSC stream is decoded (step 503) into the individual brainwaves (beta 504A, alpha 504B, delta 504C, gamma 504D, and theta 504E). As shown in the figure(s) each brainwave is associated with a different component of the final composition (e.g., music or sound). Each brainwave can be associated with a specific note or effect which is applied to another predetermined brainwave(s). Each brainwave or data stream is translated from the frequency values into a computer readable format that can be received and processed by the program 110. FIG. 5 shows a variety of different steps which can be performed to each brainwave based on if the brainwave is associated with a sound, instrument, effect, or the like. The Figure shows examples of the steps and processes for a variety of situations but can be altered based on the sound, instrument, or effect. A visual representation of the received brainwaves is shown in FIG. 6. FIG. 6 shows a user interface 600 where each of the brainwaves 601, 602, 603, 604, and 605 have a separate line to show the fluctuation in the received value. The brainwaves can be shown in a variety of formats, this is just one example of the visual representation of the brainwaves.

Each brainwave data which is collected, is translated (Step 505) to a form that is readable by the program, software, or third-party software to perform the following steps. When the brainwave signal is associated with a sound or instrument, the note(s) which are based sound or instrument have a threshold value applied to them (step 506) based on the sound or instrument, or predetermined threshold values. The threshold value is done to keep the brainwave within a set of predetermined notes. A pitch is then applied to the note (step 507), so that the note is within an aesthetically pleasing range and within a range that conforms to the rest of the brainwaves and the purpose of the overall piece. This pitch may be predetermined based on the note, the other sounds which are assigned to the other brainwaves, or by preset parameters based on the user. The velocity of the note is then calculated (step 508). The velocity may be based on the fluctuations in the brainwave frequency or other factors such as the other brainwaves calculated velocity and pitch, the purpose of the session (e.g., therapy, relaxation, live performance, etc.), or external factors such as limits set by the program 110 or manual limits set. Once the note is identified and the pitch and velocity are determined, the note is processed through a mid-stream processor (step 513). The mid-stream processor applies a series of filters (e.g., low note filter, high note filter, diatonic scale filter, etc.) to adjust the note so that in combination with the other brainwaves, the notes produced are complimentary and reasonably consonant with one another and produce a melodic piece.

Each note is then further processed to determine if the note is to remain monophonic or be manipulated to be polyphonic (step 514). The mono or polyphonic may have lead, melody, and ambiance sections or portions based on the overall design of the musical piece which is to be created and based on the program 110 desires musical composition. If the note is to be converted to polyphonic, based on the note, overall impression of the piece, and additional factors set forth by the program 110 or the user/operator, additional notes may be applied to the generated note. This is done to further improve the overall experience of the music which is produced by the program 110. A single note may not provide the desired relaxation, so based on the generated note of the brainwave, the program 110 is able to create complimentary notes to improve the music which are produced. The program 110 is able to, through pre-programing or computer learning, generating complimentary notes based on the note generated by the user's brainwave(s).

(For accuracy in this section . . . generally it is pre-determined during sound selection if the entire instrument is capable of monophonic or polyphonic function)

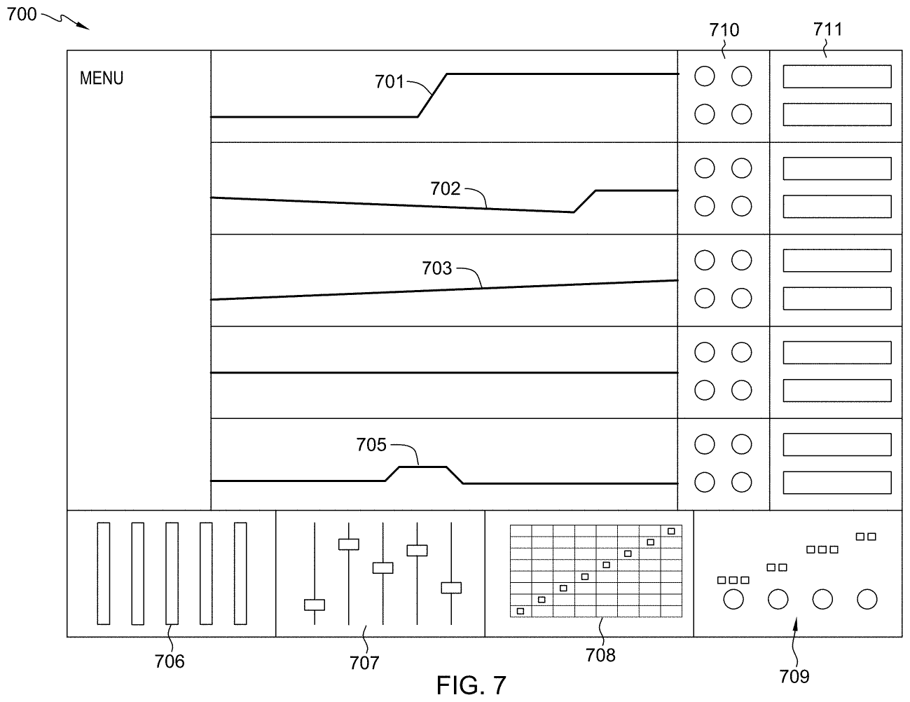

This note(s) is then electronically given an envelope (step 515) to provide the attack, decay, sustain, and release (ADSR) based on the overall impression of the created music, the specific note, the collected brainwave data, and the like. As shown in FIG. 7, the ADSR is shown for each brainwave. The ADSR is adjustable and may be manually manipulated or may be manipulated by the program 110 as new data is received. Once the note is created and the ADSR is established, additional time-based audio effects are applied to the note (step 516). The effects for example can be reverb and echo. These time-based effects are applied to the ADSR envelope to modify the ADSR envelope and modify the notes which is produced. The note is then supplied to a mixer (520) as shown in FIG. 7, which in turns sends the note to the output (step 521) to create the sound through the speakers or the like. Each brainwave 701, 702, 703, 704, and 705 is shown which is able to be independently controlled with a set of controls 710 and 711. Controls 706, 707, 708, and 709 are shown to indicate that additional controls can be incorporated based on the program or software. Various types of third-party software can be used, and this is just one example of the user interface showing the different brainwaves and the various controls.

For brainwaves 504B, 504C, 504D, and 504E the steps or processing are shown in different embodiments and methods based on the software used, and the desired end result and user involvement in the process. As shown for brainwave 504B, the parameter determination step (step 509) determines the high and low ranges and thresholds for the brainwave. In this embodiment, the program through artificial intelligence or machine learning is able to adjust and set these parameters based on the desired results and ranges. In step 510, the effect filter selection occurs. This selection of the filters and effects for the note or instrument to adhere to the purpose of the piece may be performed by machine learning technology, artificial intelligence, or manually. These steps may be performed automatically or manually based on the user preference. For brainwave 504C, the low note filter (step 517) and high note filter (Step 518) are separated out so that each note filter can be performed independent of one another. Additionally, the monophonic and polyphonic determination step (519) is shown. For brainwaves 504D and 504E, 504D is associated with a note or instrument and 504E determines the effects and filters which are applied to the adjusted note (Step 512). In step 511, the program performs a note adjustment process, wherein the note is adjusted within desired threshold values, pitch values, velocity values, and the like to meet a set of requirements and parameters set by the program or a by a user. As shown the effects are calculated and determined by 504E and applied to 504D. This shows a variety of setups for the use of the brainwaves and how the brainwaves affect one another in some cases or how the processing of the brainwaves is adjusted based on the program and third-party software which is used. In a variety of embodiments, the brainwaves may be used to affect one another or run independently of one another. The relationship between the brainwaves is determined by the user or the program.

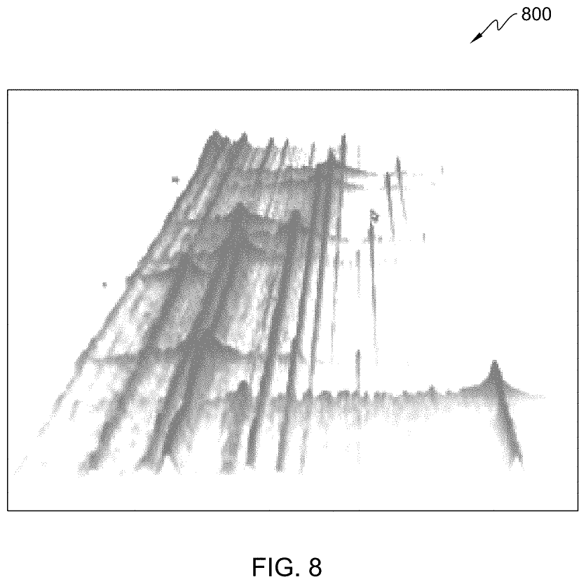

All of these creates notes are then processed thorough a mixer or program to produce the final composition in an audible format. Given the constant stream of data from the EEG machine 109 this produces a continuous piece of music (provided data is being provided by the EEG machine 109) that is adjusted based on the brain activity of the user, creating a one-of-a-kind piece of music. In some embodiments, the present invention is able to take the brainwaves and create visuals of the brainwaves as shown in FIG. 8. This shows the change in frequencies of the brainwaves. In other embodiments, these visuals can take on different embodiments than just waves.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein that are believed as maybe being new, this understanding, as indicated by use of the term “present invention,” is tentative and provisional and subject to change over the course of patent prosecution as relevant information is developed and as the claims are potentially amended.

The foregoing descriptions of various embodiments have been presented only for purposes of illustration and description. They are not intended to be exhaustive or to limit the present invention to the forms disclosed. Accordingly, many modifications and variations of the present invention are possible in light of the above teachings will be apparent to practitioners skilled in the art. Additionally, the above disclosure is not intended to limit the present invention. In the specification and claims the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

Although various representative embodiments of this invention have been described above with a certain degree of particularity, those skilled in the art could make numerous alterations to the disclosed embodiments without departing from the spirit or scope of the inventive subject matter set forth in the specification and claims. Joinder references (e.g., attached, adhered, joined) are to be construed broadly and may include intermediate members between a connection of elements and relative movement between elements. As such, joinder references do not necessarily infer those two elements are directly connected and in fixed relation to each other. Moreover, network connection references are to be construed broadly and may include intermediate members or devices between network connections of elements. As such, network connection references do not necessarily infer those two elements are in direct communication with each other. In some instances, in methodologies directly or indirectly set forth herein, various steps and operations are described in one possible order of operation, but those skilled in the art will recognize that steps and operations may be rearranged, replaced or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

Although the present invention has been described with reference to the embodiments outlined above, various alternatives, modifications, variations, improvements and/or substantial equivalents, whether known or that are or may be presently foreseen, may become apparent to those having at least ordinary skill in the art. Listing the steps of a method in a certain order does not constitute any limitation on the order of the steps of the method. Accordingly, the embodiments of the invention set forth above are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements and/or substantial equivalents.

What is claimed is:

1. A computer-implemented method comprising:
receiving, by one or more processors, data from an electroencephalogram device worn by a user, wherein data is collected related to at least one brainwave;
separating, by the one or more processors, the collected data into individual data streams related to the one or more brainwaves;
performing, by the one or more processors, at least one manipulation to each of the individual data streams, wherein each of the data streams are manipulated to produce a sound;
applying, by the one or more processors, at least one filter to each of the sounds; and
generating, by the one or more processors, each of the sounds, wherein a musical composition is formed.
The computer-implemented method of claim 1, wherein the at least one manipulation of the individual data streams, further comprising, adjusting, by the one or more processors, a note within a predetermined range.

2. The computer-implemented method of claim 1, wherein the at least one manipulation of the individual data streams, further comprising, adjusting, by the one or more processors, a note within a predetermined pitch.

3. The computer-implemented method of claim 1, wherein the at least one manipulation of the individual data streams, further comprising, adjusting, by the one or more processors, a note within a predetermined velocity.

4. The computer-implemented method of claim 1, wherein the applying at least one filter, further comprising, establishing, by the one or more processors, a note envelope.

5. The computer-implemented method of claim 4, further comprising, adjusting, by the one or more processors, an attack, a decay, a sustain, and a release of the note.

6. The computer-implemented method of claim 1, further comprising, manipulating, by the one or more processors, of the musical composition, so that the musical composition can be received by a mixer.

7. The computer-implemented method of claim 1, further comprising, switching, by the one or more processors, the note from monophonic to polyphonic.

8. A computer program product comprising:
a computer non-transitory readable storage medium having program instructions embodied therewith, the program instructions executable by a computing device to cause the computing device to:
receiving data from an electroencephalogram device worn by a user;
decoding the data received by the electroencephalogram device into individual brainwaves;
associating each of the individual brainwaves to a sound or to an effect of a sound;
performing a translation to each of the individual brainwaves collected data;
applying a plurality of manipulations to the translated data for each of the individual brainwaves;
generating a series of sounds from the manipulated translated data from the individual brainwaves associated with a sound, and wherein the individual brainwaves which are associated with an effect of a sound are applied to the corresponding manipulated translated data from the individual brainwaves associated with a sound; and
outputting, a musical composition from the series of sounds.

9. The computer program product of claim 9, wherein the effects are related to a note threshold value of the sound.

10. The computer program product of claim 9 wherein the effects are related to a pitch value of the sound.

11. The computer program product of claim 9 wherein the effects are related to a velocity value of the sound.

12. The computer program product of claim 9 further comprising, adjusting an attack, a decay, a sustain, and a release of the note.

13. The computer program product of claim 9, further comprising, playing a predetermined sound for the user to invoke certain responses for predetermined brainwaves.

14. The computer program product of claim 9, further comprising, generating a visual representation of the brainwaves.

15. A system comprising:
a CPU, a computer readable memory and a computer non-transitory readable storage medium associated with a computing device;
receiving data from an electroencephalogram device worn by a user;
decoding the data received by the electroencephalogram device into individual brainwaves;
associating a first set of brainwaves to a sound and a second set of brainwaves to an effect;
performing a translation of each of the brainwaves into data;
associating the second set of brainwaves associated with an effect to the first set of brainwaves associated with a sound;
applying the effects to the associated brainwave, wherein a modified sound is created; and
generating a musical composition from the manipulated sounds.

16. The system of claim 16, wherein the effect adjusts the sound to be within a note threshold value.

17. The system of claim 16, wherein the effect is related to the pitch and velocity of the sound.

18. The system of claim 16, further comprising manipulating an envelope of the sound.

19. The system of claim 16 further comprising, manipulating the sounds from a monophonic to a polyphonic.
